(19) United States
(12) Patent Application Publication
(10) Pub. No.: US 2023/0210979 A1
(43) Pub. Date: Jul. 6, 2023

(54) ENGINEERING BROADLY REACTIVE CORONAVIRUS VACCINES AND RELATED DESIGNS AND USES

(71) Applicant: Greffex, Inc., Houston, TX (US)

(72) Inventors: Uwe D. STAERZ, Denver, CO (US); Daniel F. PRESTON, Denver, CO (US)

(21) Appl. No.: 17/996,727

(22) PCT Filed: Apr. 20, 2021
(86) PCT No.: PCT/US2021/028187

§ 371 Date: Oct. 20, 2022

Related U.S. Application Data
(60) Provisional application No. 63/012,360, filed on Apr. 20, 2020.

(51) Int. Cl. A61K 39/215; A61P 31/14; C12N 15/86

(52) U.S. Cl. CPC A61K 39/215; A61P 31/14; C12N 15/86; C12N 2770/20071; C12N 2770/20034; C12N 2750/14143; C12N 2750/14171; A61K 2039/53

(57) ABSTRACT

A vaccine for preventing β-CoV infection includes at least one viral vector containing a β-CoV DNA sequence which codes the S protein for the β-CoV. The β-CoV RNA sequence can be a SARS-2 β-CoV DNA sequence. The vaccine may further includes a packaging plasmid based on an adenovirus. The viral vector and packaging plasmid can be contained in a packaging cell and encapsidated in a capsid. A method of vaccinating a mammal subject against infection from at least one group of β-CoV includes separating a broad group of β-CoV into homology groups based on similarities in the β-CoV RNA sequences which code for their S proteins, identifying at least one consensus sequence for each homology group which has a sequence identity of greater than 60% to all other members of the homology group, and preparing a viral vector including at least a portion of the consensus sequence from at least one homology group.

### CROSS REFERENCE TO RELATED APPLICATIONS

This application claims priority to and is a non-provisional application of Provisional Application No. 63/012,360, filed on Apr. 20, 2020, which is incorporated herein by reference in its entirety.

### FIELD

The disclosure relates to a vaccine platform for developing coronavirus vaccines, and more particularly vaccines to protect mammals from infection from β-coronaviruses. In another embodiment, the disclosure relates to methods for developing coronavirus vaccines using identified group genetic sequences.

### SUBMISSION OF SEQUENCE LISTING

The contents of the electronic submission of the text file Sequence Listing which is named “Sequence_Listing”, which was created Apr. 20, 2021, and is 54 in size, is incorporated herein by reference in its entirety.

### BACKGROUND

Coronaviruses (CoVs) are classified into four genera: alpha-, beta-, gamma- and delta-coronaviruses. β-CoVs are enveloped, positive-strand RNA viruses capable of infecting mammals, generally bats and rodents, though many β-CoVs are known to infect humans as well. Infections with CoVs in humans and animals commonly produce mild to moderate upper-respiratory tract illnesses of short duration. Exceptions are the Severe Acute Respirator Syndrome (SARS-1), the Middle East Respiratory Syndrome (MERS) and the Wuhan-originating SARS-CoV-2 (SARS-2) (also referred to as COVID-19) that are characterized by severe and often lethal symptoms. The first cases of SARS-2 infections were seen in December 2019. As of Apr. 16, 2020, there were an estimated 632,000 cases reported and an estimated 31,000 deaths in the United States alone, as reported by the Center for Disease Control (CDC), resulting in a 4.9% lethality. SARS-2 is highly infectious to humans. The World Health Organization (WHO) declared the SARS-2 worldwide pandemic a Global Health Emergency on Jan. 30, 2020.

Specific treatments for SARS-2 are not available but under investigation. The best approach to prevent further spread of the disease is the development of specific vaccines. Herd immunity against SARS-2 is better achieved with immunization with a benign vaccine rather than by the natural infection with the active SARS-2 virus. One explanation for the low-level immune response seen in recuperating patients may be a function of exhaustive immune suppression by SARS-2. However, animal studies with traditional vaccines using an inactive version of the virus have suggested that inactivated virus vaccines might be especially prone to induction of antibody dependent enhancements (ADE) of the disease. For these vaccinations, Th2-type disease enhancement may be caused by anti-nucleocapsid (NP) response. It is desirable to develop a SARS-2 vaccine which does not stimulate ADE in vaccine recipients.

While social distancing has successfully suppressed the aggressive spread of SARS-2, it is anticipated that the reopening of societies will lead to a jump in infections in short order, as well as possible seasonal occurrences. Some regions have already seen jumps in infections with mutated versions of the SARS-2 virus. The overall mutation rates of SARS-related β-CoVs (SARSrs) have been calculated at as low as 0.1 mutations per generation. Despite the recent emergence of mutations, the SARS-2 virus seems to be similarly stable. It is desirable that any SARS-2 vaccine also provide protection against short-term variants.

Numerous animal as well as clinic trials with the related SARS- and MERS-CoVs have suggested that effective vaccines could be produced against more general β-CoV infections. SARS-2 (COVID-19) is the third lethal β-CoV that has jumped from animal hosts to humans. Considering that 1,800 SARSrs have already been identified in animals, some of which may eventually infect humans, it is desirable to also create group-specific SARSr vaccines to avert future pandemics.

The use of viral vectors, including viral vectors based on the adenovirus, in vaccines is known. Such “ad vectors” repeatedly demonstrate higher and more sustained immunogenicity in comparisons to other vaccine systems. One problem with using ad vectors in vaccination programs is the strong immune response triggered against the adenovirus itself, as opposed to the target virus. To avoid these strong anti-adenovirus responses, ad vectors fully deleted (fd) of all endogenous adenovirus genes were developed. The packing information for fd adenovirus genomes was originally delivered with second viral constructs—a hybrid baculovirus-adenovirus or a helper virus. Unfortunately this led to contaminations of the replication component of the ad vector or helper viruses. It is desirable to develop an ad vector vaccine system which avoids these problems with existing ad vector vaccines.

### SUMMARY

In one embodiment, the disclosure provides a vaccine for preventing β-CoV infection. In accordance with embodiments of the present disclosure, a vaccine for preventing β-CoV infection comprises at least one viral vector comprising a β-CoV DNA sequence which codes the S protein for the β-CoV.

In an embodiment, the vector is an adenovirus vector. In another embodiment, the vector is a fully deleted adenovirus vector free of all endogenous genes. In still another embodiment, the β-CoV DNA sequence is a SARS-2 β-CoV DNA sequence. In a further embodiment, the SARS-2 β-CoV DNA sequence is the entire sequence coding the S protein. In yet a further embodiment, the SARS-2 β-CoV DNA sequence is a partial sequence coding the S protein. In another embodiment, the SARS-2 β-CoV DNA sequence is a partial sequence coding the S protein from which the receptor binding domain has been removed. In still another embodiment, the SARS-2 β-CoV DNA sequence is a partial sequence coding the S protein in which the receptor binding domain sequences have been replaced by DNA coding for a peptide linker.

In an embodiment, the vaccine further comprises a packaging plasmid based on an adenovirus selected from the group consisting of the Ad2, Ad5, Ad6 and Ad35 serotypes and combinations thereof. In a further embodiment, the at least one viral vector is contained in a packaging cell. In yet another embodiment, the packaging cell is encapsidated in a capsid selected from the group consisting of the Ad2, Ad5, Ad6 and Ad35 serotypes, and combinations thereof.

In an embodiment, the β-CoV DNA sequence is a SARS-2 β-CoV DNA sequence, and the viral vector comprises at least a second β-CoV DNA sequence from a SARSr virus, wherein the second β-CoV DNA sequence codes the S protein for the SARSr virus.

In one embodiment, the disclosure provides a vaccine for preventing SARS-2 infection. In accordance with embodiments of the present disclosure, a vaccine for preventing SARS-2 infection comprises at least one viral vector comprising a SARS-2 β-CoV DNA sequence which codes the S protein for the SARS-2 β-CoV and at least one packing plasmid based on an adenovirus selected from the group consisting of the Ad2, Ad5, Ad6 and Ad36 serotypes and combinations thereof, wherein the at least one viral vector and at least one packing plasmid are contained in a packaging cell, and wherein the packaging cell is encapsidated in a capsid selected from the group consisting of the Ad2, Ad5, Ad6 and Ad35 serotypes and combinations thereof.

In an embodiment, the SARS-2 β-CoV DNA sequence codes for a partial S protein of the SARS-2 virus.

In one embodiment, the disclosure provides a vaccine for preventing β-CoV infection. In accordance with embodiments of the present disclosure, a vaccine for preventing β-CoV infection comprises at least one β-CoV RNA sequence which codes the S protein for the β-CoV.

In an embodiment, the RNA is mRNA. In a further embodiment, the β-CoV RNA sequence is a SARS-2 β-RNA sequence. In still a further embodiment, the SARS-2 β-CoV RNA sequence is the entire sequence coding the S protein. In yet another embodiment, the SARS-2 β-CoV RNA sequence is a partial sequence coding the S protein. In still another embodiment, the SARS-2 β-CoV RNA sequence is a partial sequence coding the S protein, from which the receptor binding domain has been removed. In a further embodiment, the SARS-2 β-CoV RNA sequence is a partial sequence coding the S protein, in which the receptor binding domain sequences have been replaced by RNA coding for a peptide linker.

In an embodiment, the vaccine further comprises an expression vector that delivers the genetic information for the β-CoV RNA. In another embodiment, the expression vector is an engineered viral vector.

In one embodiment, the disclosure provides a vaccine for preventing β-CoV infection. In accordance with embodiments of the present disclosure, a vaccine for preventing β-CoV infection comprises at least one viral vector comprising a β-CoV protein sequence which codes the S protein for the β-CoV.

In an embodiment, the β-CoV RNA sequence is a SARS-2 β-CoV protein sequence. In another embodiment, the SARS-2 β-CoV protein sequence is the entire sequence coding the S protein. In still a further embodiment, the SARS-2 β-CoV protein sequence is a partial sequence coding the S protein. In still another embodiment, the SARS-2 β-CoV protein sequence is a partial S protein sequence, from which the receptor binding domain has been removed. In yet another embodiment, the SARS-2 β-CoV protein sequence is a partial S protein sequence, in which the receptor binding domain sequences have been replaced by a peptide linker.

In one embodiment, the disclosure provides a method of vaccinating a mammal subject against infection from at least one group of. β-CoV. In accordance with embodiments of the present disclosure, a method of vaccinating a mammal subject against infection from at least one group of β-CoV, the method comprises separating a broad group of β-CoV into homology groups based on similarities in the β-CoV RNA sequences which code for their S proteins; identifying at least one consensus sequence for each homology group which have a sequence identity in excess of 60% to all other members of the homology group; and preparing a viral vector including at least a portion of the consensus sequence from at least one homology group.

In an embodiment, the consensus sequence is selected from the group consisting of DNA sequences, RNA sequences, protein sequences and combinations thereof.

In an embodiment, the step of preparing of the viral vector comprising including at least a portion of a consensus sequence from two or more homology groups.

In an embodiment, the method further comprises injecting the vaccine into the mammal subject.

In one embodiment, the disclosure provides a method of vaccinating a mammal subject against infection from at least one group of β-CoV. In accordance with embodiments of the present disclosure, a method of vaccinating a mammal subject against infection from at least one group of β-CoV, the method comprises separating a broad group of β-CoV into homology groups based on similarities in the β-CoV DNA, RNA or protein sequences which code for their S proteins; identifying at least a portion of the β-CoV protein sequences for each homology group which have a sequence identity in excess of 60% to all other members of the homology group; and preparing a DNA, RNA or protein vaccine including at least a portion of the β-CoV protein sequence from at least one homology group.

In another embodiment, the method further comprises injecting the vaccine into the mammal subject.

### BRIEF DESCRIPTION OF THE DRAWINGS

FIG. 1 is a schematic showing functional portions of a SARS-2 β-CoV RNA segment which encodes the S protein, along with the portions of greatest variability and portions eliciting the greatest immune responses, in accordance with embodiments of the present disclosure.

FIG. 2 illustrates the components of a vaccine in accordance with embodiments of the present disclosure.

FIG. 3 shows the activities of an avian influenza vaccine utilizing a viral vector of the present disclosure. Specifically, FIG. 3A shows the subject groups survival rates, FIG. 3B shows the subject groups body weights, FIG. 3C shows the serum antibody titers, and FIG. 3D shows the lung virus titers.

FIG. 4 shows the activity of a MERS-CoV vaccine.

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to compass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 90° or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B; A or B; A (alone); and B (alone). Likewise, the term “and/or” as used in a phrase such as “A, B and/or C” is intended to encompass each of the following embodiments” A, B and C; A, B, or C; A or C; A or B; B or C; A and C; A and B; B and C; A (alone); B (alone); and C (alone).

In an embodiment, the present disclosure provides a method for preparing a vaccine for preventing at least one β-CoV infection in a subject, particularly a mammal subject, and more specifically a human subject.

### Identifying β-CoVs

In accordance with embodiments of the present disclosure, a method for preparing a vaccine for preventing at least one β-CoV infection in a subject, particularly a mammal subject, and more specifically a human subject, comprises identifying at least one β-CoV from an animal host, particularly a mammal host. In a particularly embodiment, the method for preparing a vaccine for preventing at least one β-CoV infection in a subject, particularly a mammal subject, and more specifically a human subject, comprises identifying at least one β-CoV from a mammal host selected from the group consisting of a bat, a rat, a human, and combinations thereof. In an embodiment, the at least one β-CoV comprises at least one SARSr. In another embodiment, the at least one β-CoV comprises at least one SARS-2 β-CoV.

### Identifying Homology Groups

In accordance with embodiments of the present disclosure, a method for preparing a vaccine for preventing at least one β-CoV infection in a subject, particularly a mammal subject, and more specifically a human subject, comprises separating identified β-CoVs, such as those identified from an animal host, into homology groups based on similarities in genetic sequence and preparing at least one consensus sequence for each homology group. The homology groups can be based on similarities in the entirety of the β-CoVs' genetic sequences, multiple portions of the β-CoVs' genetic sequences, or a single portion of the β-CoVs' genetic sequences. The genetic sequences are selected from the group consisting of DNA sequences, RNA sequences, protein sequences, and combinations thereof. It will be understood that if a single β-CoV is identified, it is the sole member of a single homology group.

In a particular embodiment, the β-CoVs comprise a plurality of SARSrs, and the plurality of SARSrs are separated into 1, or at least 2, or at least 3, or at least 4, or at least 5 homology groups. In an embodiment, the homology groups are based on at least a portion, or at least two or more portions, or all, of the genetic sequence associated with the spike protein, the SARS receptor binding domain (RBD), an envelope protein, a nucleoprotein, and combinations thereof.

In a further embodiment, at least one SARS-2 β-CoV is identified and separated into at least one homology group.

In an embodiment, within each homology group, the genetic sequences have a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the homology group.

In an embodiment, within each homology group, the genetic sequences have a sequence identity from greater than or equal to 60%, or greater than or equal to 65%, or greater than or equal to 70%, or greater than or equal to 75%, or greater than or equal to 80%, or greater than or equal to 85% to 90%, or 95%, or 96%, or 97%, or 98%, or 99%, or less than 100% to all other members in the homology group.

In an embodiment, the genetic sequences for each homology group define a distinct protein sequence for the homology group. In an embodiment, the distinct protein is selected from the group consisting of the S protein, an envelope protein, a nucleoprotein, and combinations thereof. In a further embodiment, the distinct protein is the S protein.

In a particular embodiment, a plurality of SARSrs are analyzed and separated into 5 homology groups, wherein, within each homology group, the genetic sequences have a sequence identify from greater than 65% to 99%.

An exemplary process for identifying homology groups and consensus sequences is now provided.

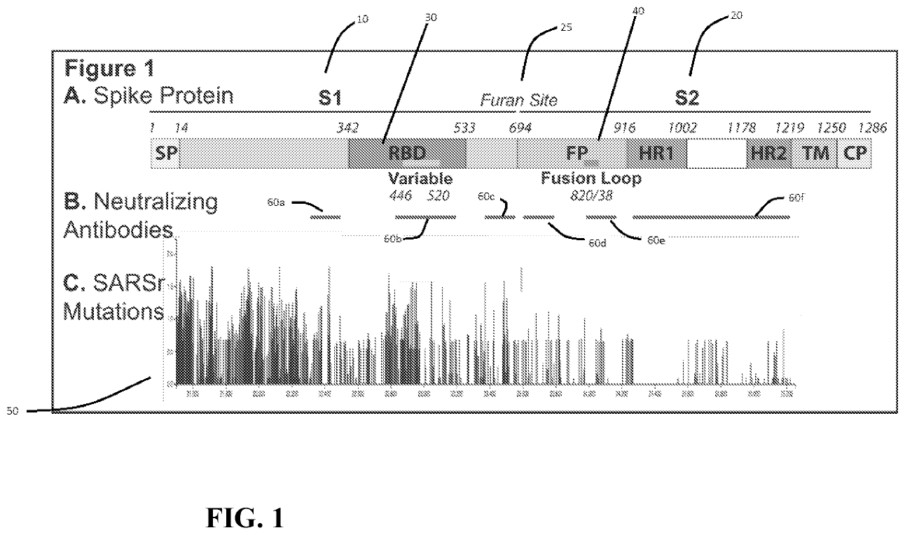

The SARS-2 β-CoV has a positive-sense, single-stranded RNA genome of about 30 kb and four structural proteins. One of the structural proteins is the spike (S) peplomer. These S proteins are found on the surface of the SARS-2 β-CoV and mediate cell receptor binding, and therefore determine the host tropism o the virus. The protein portion of the RNA which codes the S protein is divided into an S1 chain and an S2 chain, with the S1 chain 10 and the S2 chain 20 separated by a furan cut site 25, as shown in FIG. 1. The RBD 30 is located in the S1 chain 10. It was discovered that variations in the RBD influence the virus's binding to the angiotensin-converting enzyme 2 (ACE2), and an enhancement of this binding is through to facilitate the jump of the virus from animal hosts to human hosts. The membrane fusion section 40 is located in the S2 chain 20. Further shown in FIG. 1 are the heptapeptides HR1 and HR2, the transmembrane TM and the cytoplasmic domain of the S protein.

In contrast to other coronaviruses, such as the SARS-1 β-CoV, the S protein of the SARS-2 β-CoV is not enzymatically cleaved during virus assembly. The SARS-2 β-CoV S protein is pre-activated by proprotein convertase furin. Therefore, its dependence on target cell proteases on cell entry is reduced.

The SARS-2 β-CoV S protein is split into the S1 chain 10 and the S2 chain 20. Conformational changes in the S2 chain 20 lead to the fusion of the virus within the host cell. In combination with the S protein-encoding RNA sequence including the RBD, this makes the S protein-encoding RNA sequence a significant candidate for use in an anti-SARS-2 β-CoV vaccine regimen.

FIG. 1 also shows the S protein portions of the SARS-2 β-CoV which elicit greater immune responses (60). As shown, portion 60b overlaps with the RBD 30 and is a sizable portion, meaning there is significant immune response associated with the RBD 30. Portions 60d and 60e, while overlapping with the less-variable membrane fusion section 40, are smaller and therefore do not elicit as strong of an immune response. In analyzing the immune responses to different β-CoVs, such as SARS-CoV-1 and MERS-CoV, it was observed that antibodies that had the ability neutralize the activity of these coronaviruses could also bind to the more conserved areas of the S protein, namely within the S protein stem area within the S2 domain 20.

As further illustrated in FIG. 1, aligning the S protein-encoding sequences of RNA from various SARSrs shows significant divergence throughout the gene (50). A vaccine based on present SARS-2 β-CoV RNA may therefore fail to efficiently protect against infections caused by other SARSrs. However, when the S protein-encoding sequences of RNA from a plurality of SARSrs are analyzed, the SARSrs can be separated into homology groups, as shown in Table 1.

| | | | | S Protein- | |
|---|---|---|---|---|---|
| | | | Closest to | Encoding | |
| | Number of | | Consensus* | RNA | RBD |
| Group | Sequences | Consensus | Accession # | Sequence | Sequence |
| Name | Analyzed | Sequence | (GenBank) | Identity | Identity |
| All | 4276 | | SARS-2 | 68% | 69% |
| Groups | | | (QJD07688.1) | | |
| SARS- | 1130 | SEQ. ID. | AAP13441.1 | 99% | 99% |
| CoV | | 1 | | | |
| WIV-1 | 56 | SEQ. ID. | AGZ48818.1 | 97% | 92% |
| | | 2 | | | |
| Bat | 19 | SEQ. ID. | ATO98169.1 | 94% | 98% |
| 2013 | | 3 | | | |
| YNLF | 71 | SEQ. ID. | AVP78031.1 | 82% | 95% |
| | | 4 | | | |
| SARS- | 3000 | SEQ. ID. | QJD07688.1 | 99% | 98% |
| CoV-2 | | 5 | | | |

*quantified sequence most closely resembling the consensus sequence of each group - generated using EMBOSS

To obtain the information in Table 1, sequences were found using ViPR and NCBI. Global alignment was done using Clustal Omega. Related alignments (>92%) were extracted to create the groupings, which were aligned using Clustal Omega and confirmed using BLAST multi sequence alignments.

For the SARS-CoV group, 1130 sequences from GenBank and ViPR covering the original SARS-CoV-1 were analyzed. A couple of the sequences contained random inserts which are likely responsible for the gaps, but the small variants have all maintained antibody binding. For the SARS-CoV 2 group, greater than 3000 sequences were analyzed, including new clades 20H, 20I, and 20J (corresponding to the South African, California and UK variants, respectively). WIV-1 is a prominent SARSr in bats, but shown to replicate in human cells. 56 WIV-1 strains, including the RaTG13 strain thought to have given rise to SARS-2 β-CoV, were analyzed. Only 16 of the strains had complete CDS. Structures appeared steady between variants as shown by the NCBI Conserved Protein Domain Family cd21477 and Cn3D. For the YNLF group, 71 sequences (39 being complete CDS) where obtained from bats, pangolins and camels. These SARSr strains have less similarity to SARS-2 β-CoV than the WIV1 family, but have some strong similarities to the SARS-CoV group and SARS-CoV 2 group in certain regions. Global spike alignments are mediocre; however, RBD alignments show strong similarity. For the Bat2013 group, 19 samples with high similarity were analyzed. The Bat2013 group shows a higher variance than other groups, but many strains have shown cross-reactivity to the same antibodies.

### Consensus Sequence

In accordance with embodiments of the present disclosure, a method for preparing a vaccine for preventing at least one β-CoV infection in a subject, particularly a mammal subject, and more specifically a human subject, comprises identifying at least one consensus sequence for each homology group. A consensus sequence is a DNA, RNA or protein sequence developed for a group containing the statistically most frequent residue at each position in the sequence. In an embodiment, the consensus sequence for a homology group has at least 65%, or at least 70%, or at least 75%, or at least 80%, or at least 85%, or at least 90%, or at least 95%, or at least 99% commonality with each member of the corresponding homology group.

In a particular embodiment, a consensus sequence is a DNA sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In a particular embodiment, a consensus sequence is an RNA sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In a particular embodiment, a consensus sequence is a protein sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In an embodiment, the consensus sequences are edited to remove variable domains. An exemplary variable domain is shown as the sequence at 324 to 533 in FIG. 1. In such embodiments in which one or more variable domains are deleted, the deleted sequence is replaced by a smaller linker peptide designed to bridge the gap created by the deletion.

In an embodiment, the consensus sequence for each homology group is selected from the group consisting of a DNA sequence, an RNA sequence, a protein sequence, and combinations thereof. In an embodiment, the consensus sequence for at least one of the homology groups is RNA. In a further embodiment, the RNA is mRNA.

In an embodiment, the β-CoVs analyzed are SARSrs. In a further embodiment, the SARSrs include at least one SARS-2 β-CoV separated into at least one homology group, and the consensus sequence of the at least one homology group is a DNA sequence, an RNA sequence, or a protein sequence. It will be appreciated that, in embodiments wherein a single SARSr, such as a single SARS-2 β-CoV, is identified, and the single SARSr is the only member of the homology group, a consensus sequence may be a DNA sequence, RNA sequence or protein sequence will have 100% commonality with the SARSr.

In an embodiment, the consensus sequence is a SARS-2 β-CoV DNA sequence, wherein the SARS-2 β-CoV DNA sequence is at least a portion of the S protein-encoding sequences. In a further embodiment, the consensus sequence is a SARS-2 β-CoV DNA comprising the entire S protein-encoding sequence.

In an embodiment, the consensus sequence is a SARS-2 β-CoV RNA sequence, wherein the SARS-2 β-CoV RNA sequence is at least a portion of the S protein-encoding sequence.

In an embodiment, the consensus sequence is a SARS-2 β-CoV protein sequence, wherein the SARS-2 β-CoV protein sequence is at least a portion of the S protein.

### Viral Vector

In accordance with embodiments of the present disclosure, a method for preparing a vaccine for preventing at least one β-CoV infection in a subject, particularly a mammal subject, and more specifically a human subject, comprises inserting the at least one consensus sequence into a viral vector. In an embodiment, the viral vector is an adenovirus vector component.

In order to minimize pre-existing and induced interfering anti-adenovirus immune responses, all endogenous genes have been deleted from the viral vector component, which is an adenovirus vector component. That is, in an embodiment, the viral vector component is a fully deleted (fd) adenovirus vector.

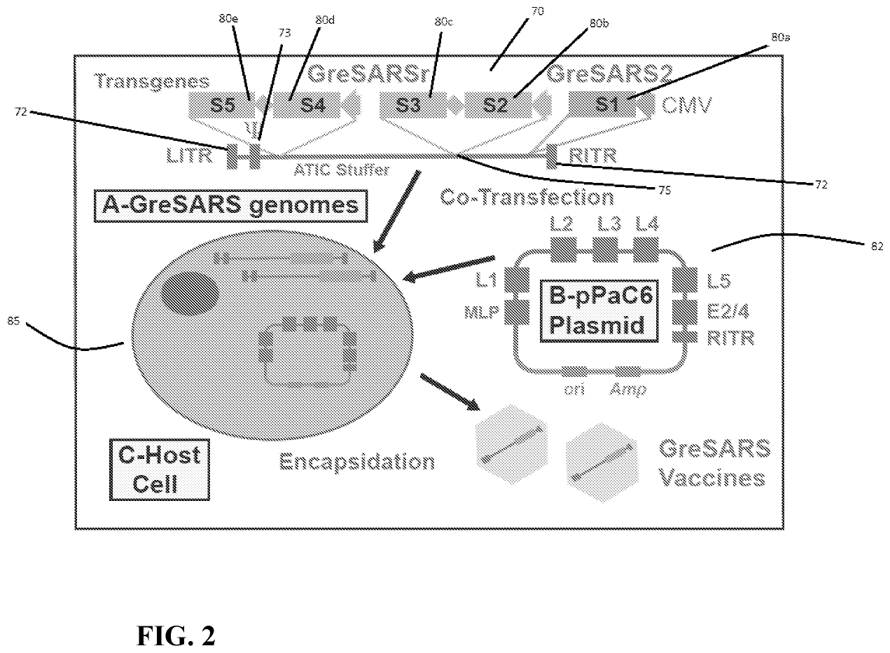

In an embodiment, the adenovirus vector 70, preferably fd adenovirus vector, is capable of receiving gene constructs of up to 33 kb and carry inverted terminal repeat sequences (ITRs) 72, 72 and a packaging signal (W) 73, as shown in FIG. 2. The deleted endogenous genes are replaced with size-compensating stuffers 75. In the embodiment shown, these stuffers 75 are prepared from fragments of the human gene 5-aminoimidazole-4-carboxamide ribonucleotide formyltrans-ferase gene (ATIC). In further embodiments, other stuffer sequences, such as, but not limited to, human hypoxanthine-guanine phosphoribosyltransferase, can be used.

In the embodiment shown in FIG. 2, five consensus sequences 80a, 80b, 80c, 80d, 80e are received by the viral vector, or adenovirus vector, or fd adenovirus vector. In further embodiments, however, a viral vector may contain more or fewer consensus sequences. A consensus sequence is in accordance with any embodiment or combination of embodiments provided herein.

For purposes of illustration only, and with reference to FIG. 2, consensus sequence 80a is a SARS-2 β-CoV RNA sequence derived from a homology group containing only SARS-2 β-CoVs. When the SARS-2 β-CoV RNA sequence is the only consensus sequence contained in the viral vector, the resulting vaccine is intended to provide specific protection from infection by SARS-2 β-CoV. In further embodiments, the consensus sequence may be derived from a homology group containing a broader collection of SARS-2 β-CoVs. In still further embodiments, the viral vector may contain additional consensus sequences, as shown in FIG. 2, derived from different homology groups. In such embodiments, the resulting vaccine may provide broader protection for viruses of the different homology groups.

In another embodiment, the viral vector may be a viral vector configured to deliver transgenes, such as DNA transgenes. Exemplary viral vectors configured to deliver transgenes include, but are not limited to Adenovirus Associated Vector and vaccinia virus vector.

### Packaging Plasmid

In accordance with embodiments of the present disclosure, a method for preparing a vaccine for preventing at least one β-CoV infection in a subject, particularly a mammal subject, and more specifically a human subject, comprises providing at least one packaging plasmid.

With further reference to FIG. 2, an exemplary plasmid 82 used in the present disclosure contains a plurality of genes, such as late genes and early genes. In an embodiment, the plasmid 82 includes a plurality of late genes, and preferably the late regions 1, 2, 3, 4 and 6 as shown in FIG. 2. In a further embodiment, the plasmid 82 used in the present disclosure contains a plurality of early genes, and preferably the early regions 2 and 4 shown in FIG. 2.

In an embodiment, the late genes and early genes are provided in trans.

As shown in FIG. 2, the plasmid 82 further includes a major late promotor (MLP) and a right ITR. The capsid used in the present disclosure is, however, void of the left ITR, the early genes E1 and E3, its packing signal, and its protein IX genes.

In a particular embodiment, the plasmid consists essentially of (i) late regions 1, 2, 3, 4, 5, (ii) early regions 2 and 4, (iii) an MLP, and (iv) a right ITR. In such an embodiment, the plasmid is wholly void of a left ITF, the early genes E1 and E3, the packing signal, and the protein IX genes.

In an embodiment, the plasmid 82 is based on an adenovirus. In a further embodiment, the plasmid 82 is based on an adenovirus selected from the group consisting of the Ad2, Ad5, Ad6 and Ad35 serotypes and combinations thereof, wherein the adenoviral capsids of the human serotype Ad2 are coded with pPaC2, Ad5 with pPaC5, Ad6 with pPaC6 and Ad35 with pPaB35. Transfection

In accordance with embodiments of the present disclosure, a method for preparing a vaccine for preventing at least one β-CoV infection in a subject, particularly a mammal subject, and more specifically a human subject, comprises transfecting a packaging cell with the viral vector(s) and packing plasmid. In an embodiment, a packaging cell may contain one or more viral vectors and one or more plasmids. In a preferred embodiment, a packaging cell comprises at least one, preferably two or more, and more preferably three or more viral vectors and one packing plasmid.

Referring still to FIG. 2, the viral vectors 70 and plasmid 82 are introduced via co-transfection into a eukaryotic host cell, or packaging cell 85. In the particular embodiment shown, the viral vectors 70 and plasmid 82 are co-transfected into the packaging cell 85 using an optimized standardized one-week co-transfection protocol.

In an embodiment, the viral vector is an adenovirus vector, particularly a fd adenovirus vector, and the packaging cell is derived from cell lines such as, but not limited to, human embryonic kidney cells (HEK293) and PerC.6 cells. The packaging cell necessary to package fd adenovirus vectors must be modified to express the genes coded within the E1 region of an adenoviral vector. In a particular embodiment, the packaging cell is an HEK293-derived Q7 packaging cell modified to express the genes coded within the E1 region of an adenoviral vector.

It is worth noting that the production of a fd adenoviral vector is initiated by the chemical transfection of the packaging cell with a mixture of the engineered adenoviral genome, the packaging expression plasmid and a chemical transfection reagent.

Capsid

In accordance with embodiments of the present disclosure, a method for preparing a vaccine for preventing at least one β-CoV infection in a subject, particularly a mammal subject, and more specifically a human subject, comprises encapsidating the packaging cell 85, containing the viral vectors 70 and plasmid 82, in a capsid, as shown in FIG. 2.

The packaging cell 85, containing the viral vectors 70 and plasmid 82, is delivered in capsids of serotypes of the adenovirus which are rare to the mammal being vaccinated. In a particular embodiment, the mammal being vaccinated is a human and the viral vector is delivered in capsids of serotypes of the adenovirus which are rare to humans. In an embodiment, the viral vector is delivered in capsids of the Ad2, Ad5, Ad6 and Ad35 serotypes, and combinations thereof. In an embodiment, the viral vector is delivered in capsids of the Ad6 serotype.

### Composition of the Vaccines

In an embodiment, the present disclosure provides a composition of a vaccine, and more particularly a vaccine to prevent against infection from β-CoVs, and preferably SARSrs.

In accordance with embodiments of the present disclosure, the vaccine includes one or more consensus sequences derived from one or more β-CoVs, and preferably one or more SARSrs, carried on at least one viral vector. A consensus sequence may be in accordance with any embodiment or combination of embodiments described herein. A viral vector may be in accordance with any embodiment or combination of embodiments described above.

In an embodiment, the one or more consensus sequences is a β-CoV DNA sequence, RNA sequence, protein sequence, or combinations thereof, and preferably a SARSr DNA sequence, RNA sequence, protein sequence, or combinations thereof.

In accordance with embodiments of the present disclosure, the one or more consensus sequences comprise at least one SARSr DNA or RNA sequence, or preferably at least one SARS-2 β-CoV DNA or RNA sequence. In an embodiment, the SARSr DNA or RNA sequence, or SARS-2 β-CoV DNA or RNA sequence, is at least a part of the S protein-encoding sequence.

In some embodiments, one or more of the one or more consensus sequences has a variable region partially or completely removed. In a particular embodiment, one or more consensus sequences comprises at least one SARSr DNA or RNA sequences, and preferably at least one SARS-2 β-CoV DNA or RNA sequence, which is at least part of the S protein-encoding sequence, and at least part of the variable region of the S protein-encoding sequences are removed.

In an embodiment, expression of the consensus sequence is driven by a promotor. The promotor may be specific to the consensus sequence, animal being vaccinated, and the particular composition of the vaccine. In an embodiment, a promotor is selected from the group consisting of human cytomegalovirus immediate early promotor/enhancer, a poly-adenylation site derived from the human growth gene, the elongation factor 1-alpha, the phosphoglycerate kinase, ubiquitin C, beta actin genes, and combinations thereof. In embodiment, the promotor's activity may be influenced by a chemical, such as, but not limited to, an antibiotic. Tetracycline is a nonlimiting example of an antibiotic that influences a promotor's activity.

In a particular embodiment, the vaccine is specifically designed to prevent infection from at least SARS-2 β-CoV. In such an embodiment, the one or more consensus sequences includes at least one SARS-2 β-CoV DNA or RNA sequence. Preferably, the SARS-2 β-CoV DNA or RNA sequence is an S protein-encoding DNA or RNA sequence. In a further embodiment, the SARS-2 β-CoV DNA or RNA sequence is an RNA sequence which is an S protein-encoding sequence (in part or in its entirety).

In an embodiment in which the consensus sequence is a SARS-2 β-CoV RNA sequence encoding the S protein (in part or in its entirety), the SARS-2 β-CoV DNA sequence is human codon-optimized and expression of the specific RNA is driven by a human cytomegalovirus immediate early promotor/enhancer followed by a poly-adenylation site derived from the human growth gene. In other embodiments, the expression of the SARS-2 β-CoV RNA is driven by other promoters, such as, but not limited to, those derived from the elongation factor 1-alpha, the phosphoglycerate kinase, ubiquitin C, beta actin genes, and combinations thereof. In another embodiment, the expression of the SARS-2 β-CoV RNA is driven by a promoter whose activity can be influenced by a chemical, such as, but not limited to, the antibiotic tetracycline.

In further embodiments, the vaccine includes two or more consensus sequences one or more viral vectors. In accordance with embodiments of the present disclosure, one consensus sequence is a SARS-2 β-CoV DNA or RNA sequence, and the vaccine includes at least one additional consensus sequence which is a SARSr DNA, RNA or protein sequence.

In an embodiment, the at least one viral vector is an adenovirus vector, and more preferably an fd adenovirus vector.

In further embodiments, the vaccine is a SARSr vaccine containing viral vectors with the SARS-2 β-CoV RNA sequence, in whole or in part) and at least one other SARSr RNA (in whole or in part) sequence. In such embodiment, the viral vector likewise carries an expression cassette of the human codon-optimized S protein for each of the SARSr groups represented on the viral vector. The human codon-optimized S protein is drive by a CMV immediate early promotor/enhancer followed by a poly-adenylation site derived from the human growth hormone.

In some embodiments, the SARS-2 β-CoV RNA and, if presented the additional SARSr RNA have had the variable region of the S protein-encoding sequences removed completely or partially.

In accordance with embodiments of the present disclosure, the vaccine further includes a packing plasmid. The packing plasmid may be in accordance with any embodiment or combination of embodiments described herein.

In an embodiment, the at least one consensus sequence is a SARSr DNA or RNA sequence, and particularly a SARSr DNA or RNA sequence which is an S protein-encoding sequence, and the packing plasmid is void of the left ITR, the early genes E1 and E3, its packing signal, and its protein IX genes. In a particular embodiment, the at least one consensus sequence is a SARSr DNA or RNA sequence, and particularly a SARSr DNA or RNA sequence which is an S protein-encoding sequence, contained on a viral vector and the packing plasmid based on an adenovirus selected from the group consisting of the Ad2, Ad5, Ad6 and Ad35 serotypes and combinations thereof, wherein the adenoviral capsids of the human serotype Ad2 are coded with pPaC2, Ad5 with pPaC5, Ad6 with pPaC6 and Ad35 with pPaB35, and the plasmid is void of the left ITR, the early genes E1 and E3, its packing signal, and its protein IX genes.

In accordance with embodiments of the present disclosure, the vaccine includes a packaging cell into which the consensus-containing viral vector(s) and plasmid(s) are co-transfected. The packaging is in accordance with any embodiment or combination or embodiments disclosed herein.

In an embodiment, the viral vector(s) and plasmid are co-transfected into the packaging cell using an optimized standardized one-week co-transfection protocol using HEK-293-derived Q7 packaging cell. In an exemplary embodiment, the viral vector contains at least one consensus sequence comprising a SARSr DNA or RNA sequence, and particularly a SARSr DNA or RNA sequence which is an S protein-encoding sequence, and plasmid is based on an adenovirus selected from the group consisting of the Ad2, Ad5, Ad6 and Ad35 serotypes and combinations thereof, wherein the adenoviral capsids of the human serotype Ad2 are coded with pPaC2, Ad5 with pPaC5, Ad6 with pPaC6 and Ad35 with pPaB35, and the viral vector(s) and plasmid are co-transfected into the packaging cell using an optimized standardized one-week co-transfection protocol using HEK-293-derived Q7 packaging cell.

The vaccine includes a capsid, in which the packaging cell (along with the viral vectors and plasmid) are encapsidated. The capsid may be in accordance with any embodiment or combination of embodiments disclosed herein.

In an embodiment, the capsid is of the Ad2, Ad5, Ad6 and Ad35 serotypes, and combinations thereof.

### Method of Vaccinating

In an embodiment the disclosure provides a method of vaccinating an animal subject, preferably a mammal subject, and more preferably a human subject against infection from at least one group of β-CoV.

In accordance with embodiments of the present disclosure, the method comprises providing a vaccine comprising at least one viral vector comprising at least one β-CoV consensus sequence, preferably at least one SARSr consensus sequence, and more preferably at least one SARS-2 β-CoV consensus sequence and a plasmid, wherein the at least one viral vector and plasmid are transfected into a packaging cell, and the packaging cell is encapsidated into a capsid.

In an embodiment, the at least one β-CoV consensus sequence is in accordance with any embodiment or combination or embodiments described herein. In an embodiment, the at least one viral vector is in accordance with any embodiment or combination of embodiments described herein. In an embodiment, the plasmid is in accordance with any embodiment or combination of embodiments described herein. In an embodiment, the packaging cell is in accordance with any embodiment or combination of embodiments described herein. In an embodiment, the capsid is in accordance with any embodiment or combination of embodiments described herein.

The method further comprising injecting the viral vector into an animal subject, preferably a mammal subject, such as, for example, a human. In an embodiment, a single dose is sufficient to provide protection against at least one β-CoV, and more specifically provide protection against any β-CoVs having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to at least one of the consensus sequences contained in the vaccine.

In other embodiments, two or more doses may be required to provide protection. In particular, two, or three, or four doses, is sufficient to provide protection against at least one β-CoV, and more particularly against any β-CoVs having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to least one of the consensus sequences contained in the vaccine.

### EXAMPLES

### Example 1

To show the efficiency of the viral vectors in accordance with embodiments of the present disclosure, BALB/c mice were given varying doses of an A/Vietname/1203/2004 (H5N1) vaccine using a viral vector in accordance with embodiments of the present disclosure and then exposed to the H5N1 virus. Particularly, there were four groups of ten mice each. A first control group (C1) is vaccinated with a placebo. A second control group (C2) is not vaccinated. A first experimental group (E1) is vaccinated with 3×10⁸ genome equivalents of the GreFluVie vaccine (containing a viral vector with a consensus sequence having at least 60% commonality with the H5N1 virus) suspended in vector suspension buffer (PBS, MgCl2 5 mM, EDTA 01 .mM, sucruose 5%). A second experimental group (E2) is vaccinated with 3×10⁷ genome equivalents of the GreFluVie vaccine suspended in vector suspension buffer. Groups C1, E1 and E2 were boosted at day 24 with the same control or vaccine preparations. On day 26, groups C1, E1 and E2 were given a medial lethal dose (LD50), applied intranasally, of H5N1. They groups were observed and their body weights determined daily. The mice were bled at day 48 and tested for the presence of antibodies neutralizing infection of MDCK test sells with the H5N1 virus and antibodies inhibiting hemagglutination horse red blood cells.

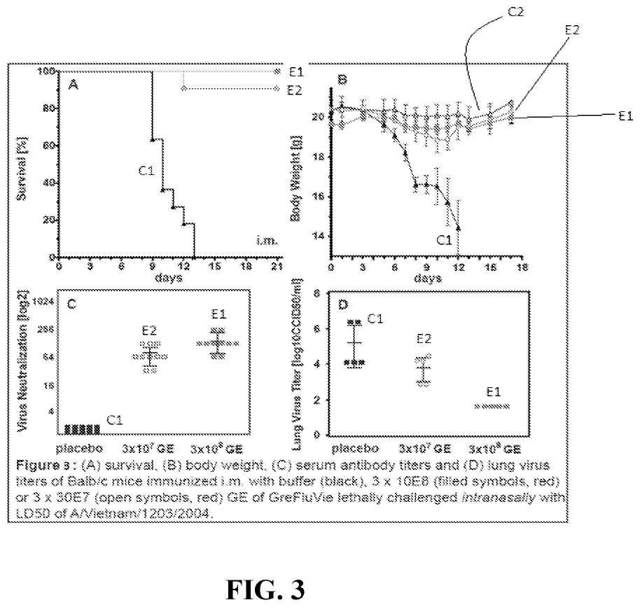

As shown in FIG. 3, the C1 group has a very low survival rate, with all mice dying before 15 days after infection. In contrast, both the E1 and E2 groups show a significantly improved survival rate, with body weights mimicking the trend of the C2 group. Of the E1 and E2 groups, the E1 group shows greater virus neutralization and a lower lung virus titer. Importantly, both the E1 and E2 groups show significant improvement in ability to fight off the infection after immunization.

### Example 2

A control group (C3) of five mice (BALB/c mice) are vaccinated with a placebo. An experimental group (E3) of five mice (BALB/c mice) are vaccinated with 3×10⁷ genome equivalents of the GreMERSfl vaccine (containing a viral vector with a consensus sequence having at least 60% commonality with the EMX/2012 MERS-CoV) suspended in a vector suspension buffer (PBS, MgCl2 5 mM, EDTA 0.1 mM, sucrose 5%). The consensus sequence is, specifically, the full-length spike protein of the MERS-CoV. Groups C3 and E3 were boosted at day 17 with the same control or vaccine preparations. On day 19 groups C3 and E3 were intranasally infected with a LD50 of MERS. The groups were blend on day 21. The sera were tested for the presence of antibodies neutralizing infection of test cells with the EMX/2012 MERS-CoV.

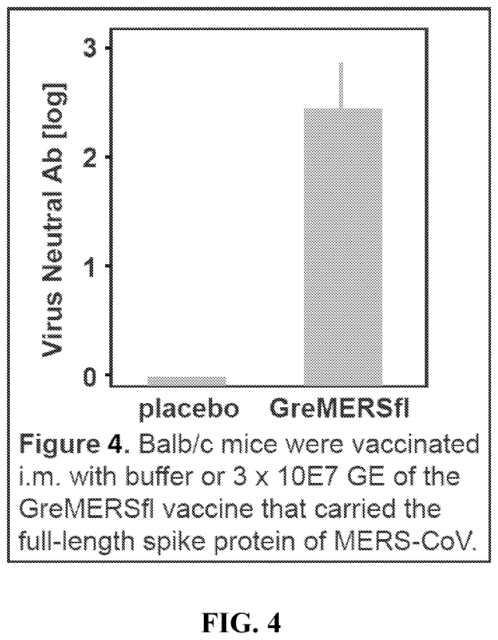

As shown in FIG. 4, group E3 showed significant improvement in virus neutralization.

While multiple embodiments of a viral vector and associated vaccine have been described in detail herein, it should be apparent that modifications and variations thereto are possible, all of which fall within the true spirit and scope of the invention. In particular, while the present viral vectors and vaccines have been described in detail with respect to β-CoVs, and more particularly SARS-2 and SARSr viruses, it will be appreciated that the viral vectors and vaccines can be modified in accordance with the skill of one in the art to apply to other classes of coronaviruses, such as, for example, α-CoVs, γ-CoVs, and δ-CoVs. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

1. A vaccine for preventing β-CoV infection, comprising:
at least one viral vector comprising a β-CoV DNA sequence which codes the S protein for the β-CoV.

2. The vaccine of claim 1, wherein the vector is an adenovirus vector.

3. The vaccine of claim 2, wherein the vector is a fully deleted adenovirus vector free of all endogenous genes.

4. The vaccine of claim 1, wherein the β-CoV DNA sequence is a SARS-2 β-CoV DNA sequence.

5. The vaccine of claim 4, wherein the SARS-2 β-CoV DNA sequence is the entire sequence coding the S protein.

6. The vaccine of claim 4, wherein the SARS-2 β-CoV DNA sequence is a partial sequence coding the S protein.

7. The vaccine of claim 4, wherein the SARS-2 β-CoV DNA sequence is a partial sequence coding the S protein from which the receptor binding domain has been removed.

8. The vaccine of claim 4, wherein the SARS-2 β-CoV DNA sequence is a partial sequence coding the S protein in which the receptor binding domain sequences have been replaced by DNA coding for a peptide linker.

9. The vaccine of claim 1, further comprising a packaging plasmid based on an adenovirus selected from the group consisting of the Ad2, Ad5, Ad6 and Ad35 serotypes and combinations thereof.

10. The vaccine of claim 1, wherein the at least one viral vector is contained in a packaging cell.

11. The vaccine of claim 10, wherein the packaging cell is encapsidated in a capsid selected from the group consisting of the Ad2, Ad5, Ad6 and Ad35 serotypes, and combinations thereof.

12. The vaccine of claim 1, wherein the β-CoV DNA sequence is a SARS-2 β-CoV DNA sequence, and the viral vector comprises at least a second β-CoV DNA sequence from a SARSr virus, wherein the second β-CoV DNA sequence codes the S protein for the SARSr virus.

13. A vaccine for preventing SARS-2 infection comprising:
at least one viral vector comprising a SARS-2 β-CoV DNA sequence which codes for the S protein of SARS-2 β-CoV, and
at least one packing plasmid based on an adenovirus selected from the group consisting of the Ad2, Ad5, Ad6 and Ad36 serotypes and combinations thereof,
wherein the at least one viral vector and at least one packing plasmid are contained in a packaging cell, and
wherein the packaging cell is encapsidated in a capsid selected from the group consisting of the Ad2, Ad5, Ad6 and Ad35 serotypes and combinations thereof.

14. A vaccine of claim 13, wherein the SARS-2 β-CoV DNA sequence codes for a partial S protein of the SARS-2 virus.

15. A vaccine for preventing β-CoV infection, comprising:
at least one β-CoV RNA sequence which codes the S protein for the β-CoV.

16. The vaccine of claim 15, wherein the RNA is a mRNA.

17. The vaccine of claim 15, wherein the β-CoV RNA sequence is a SARS-2 β-Coy RNA sequence.

18. The vaccine of claim 15, wherein the SARS-2 β-CoV RNA sequence is the entire sequence coding the S protein.

19. The vaccine of claim 15, wherein the SARS-2 β-CoV RNA sequence is a partial sequence coding the S protein.

20. The vaccine of claim 19, wherein the SARS-2 β-CoV RNA sequence is a partial sequence coding the S protein, from which the receptor binding domain has been removed.

21. The vaccine of claim 19, wherein the SARS-2 β-CoV RNA sequence is a partial sequence coding the S protein, in which the receptor binding domain sequences have been replaced by RNA coding for a peptide linker.

22. The vaccine of claim 15, further comprising an expression vector that delivers the genetic information for the β-CoV RNA.

23. The vaccine of claim 22, wherein the expression vector is an engineered viral vector.

24. A vaccine for preventing β-CoV infection, comprising:
at least one β-CoV protein sequence which codes the S protein for the β-CoV.

25. The vaccine of claim 24, wherein the β-CoV RNA sequence is a SARS-2 β-CoV protein sequence.

26. The vaccine of claim 24, wherein the SARS-2 β-CoV protein sequence is the entire sequence coding the S protein.

27. The vaccine of claim 24, wherein the SARS-2 β-CoV protein sequence is a partial sequence coding the S protein.

28. The vaccine of claim 27, wherein the SARS-2 β-CoV protein sequence is a partial S protein sequence, from which the receptor binding domain has been removed.

29. The vaccine of claim 27, wherein the SARS-2 β-CoV protein sequence is a partial S protein sequence, in which the receptor binding domain sequences have been replaced by a peptide linker.

30. A method of vaccinating a mammal subject against infection from at least one group of β-CoV, the method comprising:
separating a broad group of β-CoV into homology groups based on similarities in the β-CoV RNA sequences which code for their S proteins;
identifying at least one consensus sequence for each homology group which have a sequence identity in excess of 60% to all other members of the homology group; and
preparing a viral vector including at least a portion of the consensus sequence from at least one homology group.

31. The method of claim 30, wherein the consensus sequence is selected from the group consisting of DNA sequences, RNA sequences, protein sequences and combinations thereof.

32. The method of claim 30, wherein the preparing of the viral vector comprises preparing the viral vector including at least a portion of a consensus sequence from two or more homology groups.

33. The method of claim 30, further comprising injecting the vaccine into the mammal subject.

34. A method of vaccinating a mammal subject against infection from at least one group of β-CoV, the method comprising:
separating a broad group of β-CoV into homology groups based on similarities in the β-CoV DNA, RNA or protein sequences which code for their S proteins;
identifying at least a portion of the β-CoV protein sequences for each homology group which have a sequence identity in excess of 60% to all other members of the homology group; and
preparing a DNA, RNA or protein vaccine including at least a portion of the β-CoV protein sequence from at least one homology group.

35. The method of claim 34, further comprising injecting the vaccine into the mammal subject.


SEQUENCE LISTING (5 sequences)

SEQ ID NO: 1 - protein, 1255 aa, SARS-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 1256 aa, SARSr
MKLLVLVFATLVSSYTIEKCLDFDDRTPPANTQFLSSHRGVYYPDDIFRSNVLHLVQDHFLPFDSNVTRFITFGLNFDNPIIPFKDGIYFAATEKSNVIRGWVFGSTMNNKSQSVIIMNNSTNLVIRACNFELCDNPFFVVLKSNNTQIPSYIFNNAFNCTFEYVSKDFNLDLGEKPGNFKDLREFVFRNKDGFLHVYSGYQPISAASGLPTGFNALKPIFKLPLGINITNFRTLLTAFPPRPDYWGTSAAAYFVGYLKPTTFMLKYDENGTITDAVDCSQNPLAELKCSVKSFEIDKGIYQTSNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNTSSEVAVLYQDVNCTDVPVAIHADQLTPSWRVYSTGNNVFQTQAGCLIGAEHVDTSYECDIPIGAGICASYHTVSSLRSTSQKSIVAYTMSLGADSSIAYSNNTIAIPTNFSISITTEVMPVSMAKTSVDCNMYICGDSTECANLLLQYGSFCTQLNRALSGIAVEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGSCDVVIGIINNTVYDPLQPELDSFKEELDKYFKNHTSPDVDLGDISGINASVVNIQKEINRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 1241 aa, SARS
MKILIFAFLVTLVKAQEGCGLISRKPQPKMAQVSSSRRGVYYNDDIFRSDVLHLTQDYFLPFDSNLTQYFSLNVDSDRYTYFDNPILDFGDGVYFAATEKSNVIRGWIFGSTFDNTTQSAVIVNNSTHIIIRVCNFNLCKEPMYTVSRGTQQSSWVYQSAFNCTYDRVERSFQLDTAPKTGNFKDLREYVFKNRDGFLSVYQTYTAVNLPRGLPTGFSVLRPILKLPFGINITSYRVVMAMFSQTTSNFLPESAAYYVGNLKYTTFMLSFNENGTITDAIDCAQNPLAELKCTIKNFNVSKGIYQTSNFRVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKLSTGLVKNQCVNFNFNGLKGTGVLTDSSKRFQSFQQFGRDMSDFTDSVRDPQTLQILDITPCSFGGVSVITPGTNASSEVAVLYQDVNCTDVPTAIRADQLTPAWRVYSTGINVFQTQAGCLIGAEHVNASYECDIPIGAGICASYHTASTLRSVGQKSIVAYTMSLGAENSIAYANNSIAIPTNFSISVTTEVMPVSMSKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGRGYHLMSFPQAAPHGVVFLHVTYVPSQEKNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 4 - protein, 1246 aa, SARS
MLFFLFLQFALVNSQCVNLTGRTPLNPNYTNSSQRGVYYPDTIYRSDTLVLSQGYFLPFYSNVSWYYSLTTNNAATKRTDNPILDFKDGIYFAATEHSNIIRGWIFGTTLDNTSQSLLIVNNATNVIIKVCNFDFCYDPYLSGYYHNNKTWSIREFAVYSSYANCTFEYVSKSFMLNISGNGGLFNTLREFVFRNVDGHFKIYSKFTPVNLNRGLPTGLSVLQPLVELPVSINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSDENGVRTLSTYDFNPNVPLEYQATRVVVLSFELLNAPATVCGPKLSTQLVKNQCVNFNFNGLKGTGVLTDSSKRFQSFQQFGKDASDFIDSVRDPQTLEILDITPCSFGGVSVITPGTNTSLEVAVLYQDVNCTDVPTTIHADQLTPAWRIYATGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALSGIAIEQDKNTQEVFAQVKQIYKTPPIKDFGGFNFSQILPDPSKPSKRSFIEDLLFNKVTLADAGFIKQYGDCLGGISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYIPSQEKNFTTAPAICHEGKAHFPREGVFVSNGTHWFVTQRNFYEPKIITTDNTFVSGNCDVVIGIINNTVYDPLQPELDSFKEELDKYFKNHTSPDIDLGDISGINASVVNIQKEIDRLNEVARNLNESLIDLQELGKYEQYIKWPWYVWLGFIAGLIAIVMVTILLCCMTSCCSCLKGCCSCGSCCKFDEDDSEPVLKGVKLHYT

SEQ ID NO: 5 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT